(19) United States
(12) Patent Application Publication
(10) Pub. No.: US 2020/0368116 A1
(43) Pub. Date: Nov. 26, 2020

(54) CERAMIC BLANK FILLED WITH AN ORGANIC COMPOUND AND WITH IMPROVED MACHINING PROPERTIES

(71) Applicant: Kulzer GmbH, Hanau (DE)

(72) Inventors: Yohannes Woldegergis, Hanau (DE); Tim Weitzel, Gelnhausen (DE); Stefan Hormann, Brachttal (DE)

(73) Assignee: Kulzer GmbH, Hanau (DE)

(21) Appl. No.: 16/303,367

(22) PCT Filed: May 22, 2017
(86) PCT No.: PCT/EP2017/062270

§ 371 Date: Nov. 20, 2018

(30) Foreign Application Priority Data
May 23, 2016 (DE) 102016109437.4

(51) Int. Cl. A61K 6/818; A61C 13/00; A61C 13/083; A61K 6/889; A61K 6/891; C04B 41/00; C04B 41/48; C04B 41/83

(52) U.S. Cl. CPC A61K 6/818; A61C 13/0022; A61C 13/083; A61C 13/0018; A61C 13/0004; A61C 2201/002; A61K 6/891; C04B 41/009; C04B 41/483; C04B 41/83; A61K 6/889

(57) ABSTRACT

The invention relates to a dental ceramic blank, in particular a filled milling blank, comprising at least one organic Compound, such as a polymer polymerized in the blank, for improving the machining properties of the blank. The blank has an open-pore ceramic microstructure which has 2 to 50 wt. %, based on the total composition of the dental ceramic blank, of at least one organic Compound. The invention also relates to a method for producing the blank. The milling blank according to the invention clearly exhibits improved material properties compared to unfilled purely ceramic milling blanks which have been milled into blanks for molded prosthetic parts in CAD/CAM methods

Dental ceramic blank, in particular filled milling blank, comprising at least one organic compound, such as a polymer polymerized in the blank, for improving the processing properties of the blank, wherein the blank has an open-pored ceramic scaffold having 2 to 50% by weight of at least one organic compound, based on the total composition of the dental ceramic blank, as well as a method for the production of the blank. The milling blank according to the invention shows significantly improved material properties compared to unfilled fully ceramic milling blanks, which are milled to blanks of prosthetic molded parts in CAD/CAM-processes.

CAD/CAM processes are computer-based manufacturing processes of aesthetic prosthetic dental restorations, such as bridges and crowns. In CAD/CAM, the Englisch abbreviation CAD means Computer aided Design and CAM means Computer Aided Manufacturing.

Thus, 3D work pieces are produced in automated material-removing processes from CAD data in a CAM process. Usual removing processes comprise milling, drilling, cutting, chipping off, melting and/or at least two of the removing process steps. Beside known CAD/CAM processes, so-called laser milling, material removing caused by means of laser beams, may prospectively be used for processing of blanks for the production of prosthetic dental restorations. In order to achieve the desired results, the material properties of the blanks must be adapted specifically to the laser milling process.

The object of the invention was to develop a dental ceramic blank having improved material properties, which allows automated production of individual prosthetic molded parts from blanks. Preferably, shrink of the processed blank during sintering shall also be reduced such that formation of supporting structures, such as bars, between the milled blanks of the prosthetic molded parts and the residual blank may be largely waived. In particular, processing properties with respect to fracture strength and/or E-modulus shall be improved.

The objects of the invention were solved by the dental ceramic blank according to the invention, in particular the filled ceramic blank, according to claim 1 as well as by the method for the production of the blank according to claim 10. Preferred embodiments of the blank are illustrated in the subclaims and in the specification in more detail.

According to the invention, an open-pored ceramic matrix, synonymously to open-pored ceramic scaffold, of a partially sintered blank is infiltrated with at least one organic compound. After infiltration and solidifying of the at least one compound, the ceramic scaffold has improved material properties. Infiltration of the blank, such as the open-pored ceramic scaffold, is made in a bath of at least one liquid organic compound or organic compound being depositable from gaseous phase until complete saturation of the open-pored ceramic scaffold. Solidifying is exemplarily made by polymerization of liquid infiltrated monomer, a monomer mixture or by cooling off of a molten wax. After material processing of the blank, such as milling, the organic compound is burned out of the scaffold, i.e. the compound is pyrolised, such that the open-pored ceramic scaffold exposed again.

A subject matter of the invention is a dental ceramic blank, in particular a partially sintered blank, such as a white (white body), comprising at least one organic compound, wherein the blank has an open-pored ceramic scaffold having 2 to 50% by weight of at least one organic compound, based on the total composition of the dental ceramic blank, preferably the blank has 5 to 25% by weight, more preferably 10 to 25% by weight, particularly preferably 15 to 25% by weight of at least one organic compound, preferably HEMA, polymers of HEMA or a dental wax, based on the total composition. The blank according to the invention may also be referred to as blank filled with at least one organic compound.

The open-pored ceramic scaffold of the blank preferably has an open-pored porosity of 10 to 80%, in particular of 20 to 70%, preferably of 30 to 60%.

The open-pored ceramic scaffold of the blank preferably comprises zirconium dioxide, aluminum oxid, mixed oxide(s) comprising zirconium dioxide and zirconium dioxide and/or silicon carbide. Zirconium dioxide, in particular having a content of zirconium dioxide of greater than or equal to 50% by weight, in particular having a content of zirconium dioxide of greater than or equal to 70% by weight, is particularly preferred. Aluminum oxide having a content of greater than or equal to 95% by weight, in particular greater than or equal to 99.7% by weight, preferably greater than or equal to 99.99% by weight, is alternatively preferred. Moreover, particularly preferred ceramic scaffolds comprise a content of zirconium dioxide of greater than or equal to 50 to 100% by weight, optionally additionally comprising magnesium, such as (Mg-PSZ, partially stabilized), MgO, zirconium dioxide (Y-TZP, partially stabilized) comprising Y₂O₃, zirconium dioxide HIP state (Y-TZP, partially stabilized) comprising Y₂O₃, ZrO₂/Al₂O₃ mixed oxide, SiSiC a silicon carbide infiltrated by silicon, silicon carbide sintered without free silicon, hot-pressed silicon carbide without free silicon. Ceramic scaffolds having a content of zirconium dioxide, aluminum dioxide, mixed oxide comprising zirconium dioxide and zirconium dioxide and/or silicium carbide of greater than or equal to 70% by weight to 100% by weight, in particular greater than or equal to 73% by weight, preferably greater than or equal to 85% by weight, greater than or equal to 90% by weight, are preferred, wherein the scaffold is preferably stabilized with an yttrium compound and/or magnesium oxide.

A subject matter of the invention is a ceramic blank comprising a ceramic scaffold, in particular an open-pored ceramic scaffold, comprising a content of zirconium dioxide of 50 to 100% by weight, in particular of 70 to 100% by weight, preferably of 85% by weight to 100% by weight, particularly preferred of 90 to 100% by weight, and optionally comprising a content of other metal oxides, metalloid oxides, silicon carbide, in particular one of the afore-mentioned, or their mixtures selected from yttrium, aluminum, magnesium, potassium, calcium, lithium, and optionally silicon, having a content of 0 to 50% by weight, in particular of 0 to 30% by weight, preferably of 0 to 15% by weight, particularly preferably of 0 to 10% by weight, wherein the total composition is 100% by weight. Yttrium oxide, in particular Y₂O₃, MgO, Al₂O₃, are preferred as other metal oxides and metalloid oxides.

A particularly preferred subject matter of the invention is a ceramic blank comprising a ceramic scaffold, in particular an open-pored ceramic scaffold comprising a content of zirconium dioxide of 50 to 99.9% by weight, in particular of 70 to 99.9% by weight, preferably of 85 to 99.9% by weight, particularly preferably of 90 to 99.9% by weight or of 50 to 97% by weight, an optionally comprising a content of other metal oxides, metalloid oxides, silicon carbide, in particular one of the afore-mentioned, or mixtures thereof selected from yttrium, aluminum, magnesium, potassium, calcium, lithium, and optionally silicon, having a content of 0.1 to 50% by weight or of 3 to 50% by weight, in particular of 0.1 to 30% by weight, preferably of 0.1 to 15% by weight, particularly preferably of 0.1 to 10% by weight, wherein the total composition has 100% by weight. According to further alternatives of the invention, the ceramic scaffold comprises a feldspar or magnesium aluminate (MgAl₂O₄ spinel) or alternatively having a content of 0.001 to 5% by weight.

A particularly preferred ceramic blank according to the invention comprises a ceramic scaffold, in particular an open-pored ceramic scaffold, comprising a content of zirconium dioxide of 50 to 98% by weight or to 99.9% by weight preferably to 99.99% by weight, in particular of 70 to 98% by weight, preferably in particular of 85 to 98% by weight, as well as having a content of yttrium oxide, in particular yttrium(III) oxide of 0.01% by weight to 20% by weight, preferably of 0.1 to 15% by weight, in particular of 1 to 15% by weight, particularly preferably of 2 to 15% by weight, as well as optionally or alternatively having a content of magnesium oxide of 0.01 to 10% by weight, in particular of 0.1 to 5% b weight and/or optionally having a content of aluminum oxide, in particular Al₂O₃, of 0.01 to 30% by weight in particular of 0.1% by weight to 25% by weight, preferably the total content of the afore-mentioned components is 100% by weight. Typical ceramic scaffolds according to the invention comprise ZrO₂/Y₂O₃ having a content of 95% by weight of ZrO₂ and 5% by weight Y₂O₃ or ZrO₂/Y₂O₃/Al₂O₃ having a content of about 95% by weight ZrO₂ and about 4 to 5% by weight Y₂O₃ and about 0.25% by weight Al₂O₃; ZrO₂/Al₂O₃/Y₂O₃ having a content of about 76% by weight ZrO₂ and 20% by weight Al₂O₃ and 4% by weight Y₂O₃; ZrO₂/Y₂O₃ having a content of about 90% by weight ZrO₂ and 10% by weight Y₂O₃; ZrO₂/MgO having a content of about 96.5% by weight ZrO₂ and 3.5% by weight MgO. The above contents preferably always add to 100% by weight or optionally to 100% by weight with additives.

The at least one organic compound preferably comprises a) at least one polymerizable monomer and/or a mixture of polymerizable monomers, preferably comprising liquid, polymerizable monomers, and/or b) polymers, in particular polymers of the afore-mentioned monomers, and/or c) wax, in particular a liquefiable wax, in particular an undecomposably liquefiable wax. Sticky waxes, milling or universal waxes known by a person skilled in the art, such as in particular paraffin-containing waxes, come into consideration as wax.

The blank according to the invention may preferably be used with significantly lower material waste in an automated milling process, since the blanks significantly better withstand milling processing without chipping off of parts of the blank during the milling process due to fracture strength or flexural strength, respectively, and E-modulus. Furthermore, use of bars in the blanks between molded part and residual blank, preventing warping of the molded parts during the subsequent sintering process, may be waived more often. Usually, sintering of the molded parts is made at temperatures of 1000 to 1500° C.

Particularly preferably, the at least one monomer comprises 2-hydroxy methacrylate or a mixture with 2-hydroxy methacrylate. Further likewise preferred monomers comprise a) at least one polymerizable monomer and/or mixtures of polymerizable monomers, wherein the monomer is selected from mono-functional monomers comprising 2-hydroxyethyl methacrylate (HEMA, glycol methacrylate), alkyl methacrylate, (methyl) methacrylate and/or at least one di-, tri-, tetra- or multi-functional monomer 1,4-butanediol dimethacrylate (1,4-BDMA) or pentaerythritol tetraacrylate, bis-GMA monomer (bisphenol-A glycidyl methacrylate), triethylene glycol dimethacrylate (TEGDMA) and diethylene glycol dimethacrylate (DEGMA), tetraethylene glycol di(meth)acrylate, propoxylated neopentyl glycol diacrylate, alkyldiol di(meth)acrylate with C2 to C15 in the alkyl group, decanediol di(meth)acrylate, dodecanediol di(meth)acrylate, hexyldecanediol di(meth)acrylate, trimethylolpropane tri(meth)acrylate, pentaerythritol tetra(meth)acrylate as well as butanediol di(meth)acrylate, ethylene glycol di(meth)acrylate, polyethylene glycol di(meth)acrylate, ethoxylated/propoxylated bisphenol-A di(meth)acrylate, tris(2-hydroxyethyl)-isocyanurate triacrylate, urethane (meth)acrylate, as bis(methacryloxy-2-ethoxycarbonylamino)-alkylene, with alkylene from 2 to 15 C-atoms, UDMA, a mixture containing at least one of said (meth)acrylates and/or polymers and/or co-polymers comprising one or at least two of the afore-mentioned monomers. Methyl-, ethyl-, n-propyl-, i-propyl, n-butyl, t-butyl, i-butyl, benzyl- and furfuryl methacrylate or mixtures thereof are suitable as alkyl methacrylates. Thereof, methyl methacrylate is particularly preferred.

Particularly preferred polymers as the at least one organic compound are obtainable by polymerization of at least one of the afore-mentioned monomers or a mixture comprising at least two of the afore-mentioned monomers.

Moreover, thermal initiators, such as peroxides or azo compounds, may be added to the monomers.

The wax may preferably be selected from dental waxes, in particular comprising paraffins, ceresin, carnauba wax, cacao butter, beeswax, stearic acid and/or microcrystalline paraffinic hydrocarbon waxes. Preferably, the wax has a melting temperature of greater than or equal to 140° C., in particular greater than or equal to 145° C. Those high-melting-point waxes are particularly well suited for infiltration of milling blanks. Alternatively, the wax may have a melting temperature of greater than 50° C. to 100° C. or of greater than 100° C. to 139° C.

Preferably, the blank according to the invention is a white (partially sintered blank), a green body or a hot-isostatically pressed (HIP) blank, preferably the blank is a white.

Furthermore, the blank according to the invention preferably has a fracture strength or flexural strength, respectively, according to EN DIN 6872:2008 of greater than or equal to 41 N/mm² and/or an E-modulus of greater than or equal to 37 kN/mm², measured by 4-point bending test DIN EN ISO 6872:2008. Moreover, a blank having at least one polymer as organic compound has a fracture strength of greater than or equal to 60 N/mm², in particular of greater than or equal to 70 N/mm², greater than or equal to 80 N/mm², greater than or equal to 90 N/mm², greater than or equal to 95 N/mm², in particular greater than or equal to 100 N/mm², and optionally to 110 N/mm² and/or an E-modulus of greater than or equal to 37 kN/mm², in particular greater than or equal to 40 kN/mm², preferably greater than or equal to 45 kN/mm², particularly preferably greater than or equal to 50 kN/mm², preferably greater than or equal to 55 kN/mm², more preferably of greater than or equal to 60 kN/mm² measured by 4-point bending test DIN EN ISO 6872:2008. Blanks having a fracture strength or flexural strength, respectively, of greater than or equal to 60 N/mm², in particular greater than or equal to 70 N/mm², preferably greater than or equal to 80 N/mm², greater than or equal to 90 N/mm², in particular greater than or equal to 100 N/mm² and having an E-modulus of greater than or equal to 40 kN/mm², preferably greater than or equal to 50 kN/mm² measured by 4-point bending test DIN EN ISO 6872:2008, are particularly preferred.

The blank according to the invention is particularly well suited for the production of at least one prosthetic dental or medical blank of a molded part, such as a white, in a material-removing process, in particular in a CAD/CAM-process, comprising milling, drilling and/or cutting processing and/or material-removing processing by means of laser. Consequently, a subject matter of the invention are milling blanks and blanks for material-removing processing by means of laser. The whites of dental molded parts are subsequently sintered.

Likewise a subject matter of the invention is a method for the production of a blank as well as a blank obtainable according to the method, comprising at least one organic compound, in which (i) a blank having an open-pored ceramic scaffold is contacted with at least one liquid organic compound or organic compound being depositable from gaseous phase, and (ii) the open-pored ceramic scaffold of the blank incorporated 2 to 50% by weight of at least one organic compound, based on the total composition of the dental ceramic blank.

In this context, it is preferred for the blank to be infiltrated by at least one liquid organic compound or at least one organic compound being depositable from gaseous phase in which the blank is condensed.

The method step (i), in which the blank having an open-pored ceramic scaffold is contacted with at least one liquid organic compound or at least one organic compound being depositable from gaseous phase, may be made by infiltration under pressure of 1.1 to 10 kbar, preferably of 2 to 10 bar, or alternatively the open-pored scaffold is previously evacuated, in particular by the open-pored scaffold being contacted with at least one liquid organic compound or organic compound being depositable from gaseous phase in vacuum at 10⁻⁵ bar to 0.9 bar, in particular 10⁻³ bar to 0.1 bar.

Furthermore, it is preferred for the open-pored ceramic scaffold of the blank to have an open-pored porosity of 10 to 80%, in particular an open-pored porosity of 20 to 70%, preferably of 20 to 50%, alternatively an open-pored porosity of approx. 25% +/−5%.

The afore-mentioned organic compound comprise the afore-mentioned a) at least one polymerizable monomer and/or mixtures of polymerizable monomers, and/or b) polymers, in particular of the monomers from a), and/or c) at least one wax.

According to the invention, it is thereby preferred for the organic compound to comprise at least one monomer and (ii) to be polymerized, or for the organic compound to comprise a liquid wax, which is (iii) solidified, in particular cooled off.

Another subject matter of the invention is the use of a liquid, polymerizable monomer, a mixture of polymerizable monomers and/or a wax, as listed above, for infiltration of an open-pored dental ceramic, in particular of an open-pored ceramic scaffold of the blank, in particular for the production of a blank filled with at least one organic compound.

Suitable initiators are a) peroxide and/or azo compound, in particular LPO: dilauroylperoxide, BPO: dibenzoylperoxide, t-BPEH: tert-butylper-2-ehtylhexanoate, AIBN: 2,2′-azobis-(isbutyronitrile), DTBP: di-tert-butylperoxide, and, optionally, b) at least one activator, in particular at least one aromatic amine, such as N,N-dimethyl-p-toluidine, N,N dihydroxyethyl-p-toluidine and/or p-dimethylamino-benzoic acid diethylester, or at least one initiator system selected from redox systems, in particular a combination selected from dibenzoylperoxide, dilauroylperoxide, and camphorquinone with amines selected from N,N dimethyl-p-toluidine, N,N-dihydroxyethyl-p-toluidine, and p-dimethylamino-benzoic acid diethylester, or a redox system comprising a peroxide, and a reduction agent selected from ascorbic acid, ascorbic acid derivative, barbituric acid or a barbituric acid derivative, sulfinic acid, sulfinic acid derivative, particularly preferred is a redox system comprising (i) barbituric acid or thiobarbituric acid or a barbituric acid derivative or thiobarbituric acid derivative, and (ii) at least one copper salt or one copper complex, and (iii) at least one compound having an ionic halogen atom, particularly preferred is a redox system comprising 1-benzyl-5-phenylbarbituric acid, copper acetylacetonate and benzyldibutylammoniumchloride. Particularly preferably, the polymerisation in the two-component prosthetic base material is started by a barbituric acid derivative.

In the following, the exemplary embodiments of the invention are explained with reference to schematically illustrated figures and exemplary embodiments, however without limiting the invention. In this context,

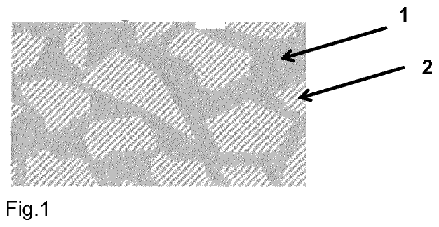

FIG. 1: schematically shows an open pored ceramic scaffold 2 filled with an organic compound 1. The organic compound is pyrolised during sintering at 1000 to 1500° C., such that the open-pored ceramic scaffold substantially remains without organic compound.

Zirconium oxide in white (white body) state (open porosity) was infiltrated by a curable liquid. For this purpose, different infiltration materials were tested, wherein monomer HEMA was used with 99.5% by weight and Interox TBPEH with 0.5% (liquid peroxide). The largest increase in strength could be achieved with this monomer, as shown by 4-point bending test according to DIN EN ISO 6872:2008.

A zirconium oxide blank having a diameter of 100 mm and a height of 14 mm was infiltrated with HEMA. Infiltration of the blank is made in a bath of the respective liquid or organic compound until complete saturation of the open-pored ceramic scaffold. The blank was laid into the monomer and subsequently cured at 90° C.

Infiltration time: 2 h; Curing time at 90° C.: 3 h

Weight difference:

| | White | Infiltrated |
|---|---|---|
| Blank | state | white state |
| 1 | 342.010 g | 387.610 g |
| 2 | 341.776 g | 380.559 g |

Flexural strength according to EN DIN 6872:2008:

### EXAMPLE 1

ZrO₂ translucent white, milled samples

Unsintered, infiltrated rose

TABLE 1a

| | Sample | | | Fracture | E- |
|---|---|---|---|---|---|
| | thick- | Sample | | strength | mod. |
| | ness | width | F max | N/ | kN/ |
| No. | mm | mm | N | mm² | mm² |
| 1 | 3.001 | 4.004 | 117.8 | 73.53 | 43 |
| 2 | 3.006 | 4.002 | 145.8 | 90.69 | 60 |
| 3 | 3.006 | 4.003 | 127.0 | 78.98 | 56 |
| 4 | 3.006 | 4.002 | 141.1 | 87.73 | 56 |
| 5 | 3.005 | 4.004 | 143.3 | 89.09 | 57 |
| 6 | 3.000 | 4.004 | 123.1 | 76.58 | 52 |
| 7 | 3.006 | 4.005 | 114.4 | 71.14 | 54 |
| 8 | 3.006 | 4.005 | 132.7 | 82.48 | 52 |
| 9 | 3.006 | 4.005 | 134.9 | 83.87 | 52 |
| 10 | 3.005 | 4.006 | 162.3 | 100.85 | 62 |
| 11 | 3.005 | 4.006 | 1339 | 83.26 | 54 |
| 12 | 3.006 | 4.004 | 146.8 | 91.28 | 56 |
| 13 | 3.005 | 4.003 | 111.0 | 69.06 | 51 |
| 14 | 3.005 | 4.004 | 169.2 | 105.28 | 40 |

TABLE 1b

| | | Fracture | |
|---|---|---|---|
| Series | F max | strength | E-mod. |
| n = 14 | N | N/mm² | kN/mm² |
| X | 135.9 | 84.56 | 53 |

### EXAMPLE 2

ZrO₂ translucent white, milled samples.

Unsintered, infiltrated white

TABLE 2a

| | Sample | Sample | F | Fracture | E-mod. |
|---|---|---|---|---|---|
| | thickness | width | max | strength | kN/ |
| No. | mm | mm | N | N/mm² | mm² |
| 1 | 3.006 | 4.005 | 153.3 | 95.33 | 62 |
| 2 | 3.002 | 4.003 | 165.0 | 102.89 | 59 |
| 3 | 3.006 | 4.005 | 164.6 | 102.35 | 56 |
| 4 | 3.005 | 4.005 | 167.5 | 104.24 | 60 |
| 5 | 3.006 | 4.004 | 145.2 | 90.27 | 58 |
| 6 | 3.006 | 4.004 | 163.5 | 101.68 | 58 |
| 7 | 3.006 | 4.006 | 145.8 | 90.60 | 59 |
| 8 | 3.000 | 4.006 | 171.3 | 106.43 | 58 |
| 9 | 3.006 | 4.006 | 125.8 | 78.17 | 60 |
| 10 | 3.005 | 4.005 | 138.3 | 86.02 | 59 |
| 11 | 3.007 | 4.003 | 159.5 | 99.17 | 59 |
| 12 | 3.003 | 4.005 | 133.2 | 82.97 | 58 |
| 13 | 3.003 | 4.005 | 124.6 | 77.60 | 55 |
| 14 | 3.005 | 4.005 | 161.4 | 100.43 | 54 |

TABLE 2b

| | | Fracture | |
|---|---|---|---|
| Series | F max | strength | E-mod. |
| n = 14 | N | N/mm² | kN/mm² |
| X | 151.4 | 94.15 | 58 |

### EXAMPLE 3

ZrO₂ translucent white, milled samples.

Unsintered, untreated, wax

TABLE 3a

| | Sample | Sample | F | Fracture | E-mod. |
|---|---|---|---|---|---|
| | thickness | width | max | strength | kN/ |
| No. | mm | mm | N | N/mm² | mm² |
| 1 | 3.026 | 4.068 | 76.8 | 46.42 | 37 |
| 2 | 3.019 | 4.070 | 71.9 | 43.58 | 39 |
| 3 | 3.028 | 4.088 | 74.5 | 44.73 | 39 |
| 4 | 3.026 | 4.061 | 74.2 | 44.88 | 40 |
| 5 | 3.023 | 4.063 | 67.7 | 41.03 | 40 |
| 6 | 3.025 | 4.091 | 81.7 | 49.09 | 40 |
| 7 | 3.040 | 4.063 | 81.9 | 49.09 | 40 |
| 8 | 3.012 | 4.065 | 78.8 | 47.59 | 37 |
| 9 | 3.016 | 4.078 | 72.0 | 43.69 | 38 |
| 10 | 3.021 | 4.080 | 67.9 | 41.02 | 37 |
| 11 | 3.027 | 4.083 | 70.9 | 42.64 | 37 |
| 12 | 3.025 | 4.067 | 79.8 | 48.23 | 35 |

TABLE 3b

| | F | Fracture | E-mod. |
|---|---|---|---|
| Series | max | strength | kN/ |
| n = 12 | N | N/mm² | mm² |
| X | 74.8 | 45.17 | 38 |

### EXAMPLE 4

### Comparative Example (Non-Infiltrated)

ZrO₂ translucent white, milled samples.

Unsintered, untreated.

TABLE 4a

| | Sample | Sample | F | Fracture | E-mod. |
|---|---|---|---|---|---|
| | thickness | width | max | strength | kN/ |
| No. | mm | mm | N | N/mm² | mm² |
| 1 | 3.018 | 4.068 | 64.4 | 39.13 | 36 |
| 2 | 3.020 | 4.068 | 67.9 | 41.17 | 35 |
| 3 | 3.021 | 4.077 | 67.2 | 40.64 | 35 |
| 4 | 3.023 | 4.070 | 62.9 | 38.05 | 36 |
| 5 | 3.006 | 4.081 | 66.9 | 40.79 | 36 |
| 6 | 3.019 | 4.062 | 66.3 | 40.26 | 35 |
| 7 | 3.024 | 4.062 | 63.5 | 38.46 | 35 |
| 8 | 3.022 | 4.065 | 69.7 | 42.24 | 35 |
| 9 | 3.014 | 4.069 | 65.6 | 39.96 | 36 |
| 10 | 3.023 | 4.067 | 69.4 | 42.04 | 35 |
| 11 | 3.020 | 4.067 | 67.3 | 40.88 | 35 |
| 12 | 3.015 | 4.067 | 62.4 | 37.96 | 35 |
| 13 | 3.018 | 4.072 | 66.8 | 40.50 | 35 |
| 14 | 3.024 | 4.060 | 67.7 | 41.04 | 35 |

TABLE 4b

| | F | Fracture | E-mod. |
|---|---|---|---|
| Series | max | strength | kN/ |
| n = 14 | N | N/mm² | mm² |
| X | 66.3 | 40.22 | 35 |

1. A dental ceramic blank comprising at least one organic compound, wherein the dental ceramic blank comprises an open-pored ceramic scaffold comprising zirconium dioxide, aluminum oxide, a mixed oxide comprising zirconium dioxide, and/or silicon carbide and comprises from 2 to 50% by weight of at least one organic compound based on the total composition of the dental ceramic blank, and further wherein the at least one organic compound is selected from the group consisting of:
(a) at least one polymerizable monomer and/or mixture of polymerizable monomers, wherein the monomer is selected from mono-functional monomers comprising 2 hydroxyethyl methacrylate (HEMA, glycol methacrylate), alkyl methacrylate, (methyl) methacrylate and/or at least one di-, tri-, tetra- or multi-functional monomer 1,4 butanediol dimethacrylate (1,4-BDMA) or pentaerythritol tetraacrylate, bis GMA monomer (bisphenol-A glycidyl methacrylate), triethylene glycol dimethacrylate (TEGDMA) and diethylene glycol dimethacrylate (DEGMA), tetraethylene glycol di(meth)acrylate, propoxylated neopentyl glycol diacrylate, alkyldiol di(meth)acrylate with C2 to C15 in the alkyl group, decanediol di(meth)acrylate, dodecanediol di(meth)acrylate, hexyldecanediol di(meth)acrylate, trimethylolpropane tri(meth)acrylate, pentaerythritol tetra(meth)acrylate as well as butanediol di(meth)acrylate, ethylene glycol di(meth)acrylate, polyethylene glycol di(meth)acrylate, ethoxylated/propoxylated bisphenol-A di(meth)acrylate, tris(2-hydroxyethyl)-isocyanurate triacrylate, urethane (meth)acrylate, as bis(methacryloxy-2-ethoxycarbonylamino)-alkylene, with alkylene from 2 to 15 C-atoms, UDMA, a mixture containing at least one of said (meth)acrylates;
(b) polymers obtainable by polymerization of at least one of the aforementioned monomers or of a mixture comprising at least two of the aforementioned monomers; and
(c) a wax, wherein the wax comprises a dental wax comprising paraffin, ceresin, carnauba wax, cacao butter, beeswax, stearic acid, and/or a microcrystalline, paraffinic hydrocarbon wax.

2-4. (canceled)

5. The dental ceramic blank of claim 1, wherein the dental ceramic blank comprises 5 to 25% by weight of at least one organic compound based on the total composition.

6. The dental ceramic blank of claim 1, wherein the dental ceramic blank is a white, green body or hot-isostatically pressed (HIP) blank.

7. The dental ceramic blank of claim 1, wherein the dental ceramic blank has a fracture strength of greater than or equal to 41 N/mm² and/or an E-modulus of greater than or equal to 37 kN/mm², measured by 4-point bending test DIN EN ISO 6872:2008.

8. The dental ceramic blank of claim 1, wherein the dental ceramic blank is suitable for the production of at least one prosthetic dental or medical blank of a molded part in a material-removing process comprising milling, drilling,. and/or cutting processing and/or material-removing processing employing a laser.

9. The dental ceramic blank of claim 1, wherein the open-pored ceramic scaffold has an open-pored porosity of 10 to 80.

10. A method for producing the dental ceramic blank of claim 1, the method comprising:
(a) contacting a dental ceramic blank comprising an open-pored ceramic scaffold comprising zirconium dioxide, aluminum oxide, or a mixed oxide comprising zirconium dioxide and/or silicon carbide with at least one liquid organic compound or organic compound being depositable from gaseous phase, the at least one organic compound selected from:
(i) at least one polymerizable monomer and/or mixture of polymerizable monomers, wherein the monomer is selected from mono-functional monomers comprising 2 hydroxyethyl methacrylate (HEMA, glycol methacrylate), alkyl methacrylate, (methyl) methacrylate and/or at least one di-, tri-, tetra- or multi-functional monomer 1,4 butanediol dimethacrylate (1,4-BDMA) or pentaerythritol tetraacrylate, bis GMA monomer (bisphenol-A plycidyl methacrylate), triethylene glycol dimethacrylate (TEGDMA) and diethylene glycol dimethacrylate (DEGMA), tetraethylene glycol di(meth)acrylate, propoxylated neopentyl glycol diacrylate, alkyldiol di(meth)acrylate with C2 to C15 in the alkyl group, decanediol di(meth)acrylate, dodecanediol di(meth)acrylate, hexyldecanediol di(meth)acrylate, trimethylolpropane tri(meth)acrylate, pentaerythritol tetra(meth)acrylate as well as butanediol di(meth)acrylate, ethylene glycol di(meth)acrylate, polyethylene glycol di(meth)acrylate, ethoxylated/propoxylated bisphenol-A di(meth)acrylate, tris(2-hydroxyethyl)-isocyanurate triacrylate, urethane (meth)acrylate, as bis(methacryloxy-2-ethoxycarbonylamino)-alkylene, with alkylene from 2 to 15 C-atoms, UDMA, a mixture containing at least one of said (meth)acrylates;
(ii) polymers obtainable by polymerisation of at least one of the afore-mentioned monomers or of a mixture comprising at least two of the afore-mentioned monomers; and
(iii) wax, wherein the wax comprises a dental wax comprising paraffin, ceresin, carnauba wax, cacao butter, beeswax, stearic acid and/or microcrystalline, paraffinic hydrocarbon waxes; and
(b) incorporating 2 to 50% by weight of at least one organic compound or organic compound based on the total composition of the dental ceramic blank into the open-pored dental ceramic scaffold.

11. The method of claim 10, wherein the dental ceramic blank is infiltrated with at least one liquid organic compound or at least one organic compound depositable from gaseous phase is condensed in the blank.

12. The method of claim 10, wherein the open-pored ceramic scaffold of the dental ceramic blank has an open-pored porosity of 10 to 80% and is infiltrated with at least one liquid organic compound or at least one organic compound depositable from gaseous phase is condensed in the blank.

13. (canceled)

14. The method of claim 10, wherein the organic compound is polymerized or the wax is solidified.

15. A dental ceramic blank produced by the method of claim 10.

16-17. (canceled)

18. The dental ceramic blank of claim 6, wherein the dental ceramic blank is a white.

19. The dental ceramic blank of claim 8, wherein the dental ceramic blank is suitable for the production of at least one prosthetic dental or medical blank of a molded part in a material-removing process employing a CAD/CAM-process comprising milling, drilling, and/or cutting processing and/or material-removing processing employing a laser.

20. The dental ceramic blank of claim 9, wherein the open-pored ceramic scaffold has an open-pored porosity of 20 to 70%.

21. The dental ceramic blank of claim 9, wherein the open-pored ceramic scaffold has an open-pored porosity of 20 to 60%.
